(19) United States
(12) Patent Application Publication
(10) Pub. No.: US 2018/0374144 A1
(43) Pub. Date: Dec. 27, 2018

(54) ON-LINE SHOPPING SYSTEM AND METHOD FOR FRESH FOOD SHOPPING

(71) Applicant: Eyepick LLC, Monroe, NY (US)

(72) Inventors: Shimon SMILOWITZ, Brooklyn, NY (US); Mendel GRATT, Spring Valley, NY (US)

(21) Appl. No.: 15/478,423

(22) Filed: Apr. 4, 2017

Related U.S. Application Data
(60) Provisional application No. 62/317,939, filed on Apr. 4, 2016.

(51) Int. Cl. G06Q 30/06; G07F 11/16; G01N 33/02; B25J 9/16

(52) U.S. Cl. CPC G06Q 30/0643; G07F 11/165; G06Q 30/0625; B25J 9/1687; G01N 33/025

(57) ABSTRACT

An online vending system for vending physically any unique food items. The vending system has a food rack system and individual robots for carrying the food items to and from the food rack system. Each food item is characterized by obtaining for it at least its image taken from plural sides thereof and a customer interface is provided that allows a customer to view and obtain various parameter data relating to each physically unique food item, to enable individual selection of fresh fruit items such as apples, pears and the like.

### BACKGROUND OF THE INVENTION

The present invention is directed to an online shopping system and method and, more particularly, to an online system that is particularly suited for the vending and purchasing fresh food products, such as fruits, vegetables, poultry, meat pieces and the like.

During the past decade, on-line shopping for various items has enjoyed an explosive growth. More and more people find it far more convenient to engage in shopping activities from the comfort of their homes and offices and subsequently have their purchased items delivered to their doorsteps. This is not particularly difficult or challenging for products such as books, electronics, furniture and even packaged foods such as wines, liquors, boxed cereal and the like which are characterized in that it is sufficient to identify the particular item by its brand name, etc. to obtain the identical product, no matter when the product is purchased and under what circumstances.

This is not so with respect to the purchasing of fresh food products such as vegetables and fruits, for example apples, oranges, tomatoes, potatoes and the like, where the typical shopper prefers to physically view each food item with their eyes from all sides thereof, sometimes even feel it or smell it and only then effect a purchasing decision. In the description below reference will be made to “physically unique food items” (“FUFI”). This phrase denotes herein non-processed food items which are inherently different from each other. No two apples or two bananas are “identical.” The same is true of cut pieces of poultry or meat. Each is unique. In the case of meat, the meat in one package has a “color” different than the other. Or, its “fat” content, e.g. marbleized appearance, differs from the next meat package. For brevity, the term “food item” refers herein to physically unique food items, such as fresh food and the like.

Conventional online shopping is not suited for such a purchasing process. To the present inventor's knowledge, prior art online vending systems sell goods based on their SKUs, i.e. item model number, and the items under a given unique SKU are all fungible with respect to each other. At most, the prior art has provided “sell by” labels on milk and similar fresh products. But this is not related to online selling.

The prior art has deemed it too impractical and technologically challengeable to be able to replicate in an online vending system the personal experience involved in shopping for fresh food (FUFIs) which allows the buyer to personally view, touch, squeeze and even smell or feel of each food item, which has not been available in the prior art online vending systems. At best, the prior art has provided information about fresh food or described methods of storing fresh food to preserve its freshness, as reflected, for example, in United States Patent Publications US-2006/0247967; US-2011/0258130; US-2009/0196969; and US-2016/0123935. The contents of the aforementioned U.S. patent publications (some of the teachings of which can be deployed in the system of the present invention) are incorporated in reference herein.

In general, an objective of the present invention is to provide a system and method that enables purchasers to purchase and vendors to vend fresh food items such as vegetables, fruits, poultry and meat pieces and the like from the convenience of their home, in a manner that does not impose on the vendor extraordinary financial or technical challenges and which replicates for the buyers the in-person experience of shopping for fresh food and the like.

### SUMMARY OF THE INVENTION

Accordingly, it is an object of the present invention to provide a system and method that overcomes the aforementioned drawbacks of the prior art.

It is another object of the invention to provide a system and method for the vending of fresh fruit products in a manner that is convenient and economical for vendors to implement, and useful for purchasers to obtain the actual food items that they have personally chosen on the given on-line web portal. If they selected a given apple, they will be shipped that apple and no other.

The foregoing and other objects of the present invention are realized in accordance with the present invention in a system and method that preferably comprises an online vending system for vending physically unique food items (FUFI). The vending system includes: a food rack system configured to hold a changing inventory of the physically unique food items, said food rack system being configured to store therein a plurality of said food items in a configuration that allows on-going replenishment of said food items; a first system for conveying the food items to and from the rack system; a second system for retrieving and shipping customer-selected ones of said food items for being shipped to customers; a processor for controlling an overall operation sequence of said online-vending system, said processor being configured to provide to remote customer electronic devices at least one customer display of said food items, each food item being displayed individually and having parameter data pertaining specifically to said each food item displayed relative to said each item, in a manner that enables the customers to select desire ones of said food items shown on said customer display and based on said parameter data; a processing facility that enables customers to communicate, via a user interface, to the processor to select specific categories of the food items to be viewed on a screen of the customer's electronic device and to thereafter select specific food items based on said parameter data provided on the screen relative to each food item, said parameter data visually or alphanumerically identifying one or more of a visual appearance, color, size, price/weight and/or smell/flavor data for each of said food items; and a robotic system for receiving various food items selected by a customer and for controlling the packaging and the shipping thereof via said second system to the customer.

Preferably, the system includes individual containers for carrying therein each of the physically unique food items and a plurality of individual robots each configured to hold one of the containers and to convey each container to and from the rack system based on receiving specific instructions from the processor. The rack system comprises a plurality of vertically stacked platforms, each platform having a floor space for the individual robots to travel thereon and including a stacking ceiling into which the containers carried by the individual robots can be inserted by being raised and attached to said ceiling, and said individual robots being able to travel on ramps to reach any of said platforms.

Preferably, the system includes a tool for holding a food item and being able to rotate and change the orientation of the food item and including an imaging system for photographing the food item with said rotating device, and a gripping mechanism and an imaging method mechanism and the gripping mechanism being configured to raise individual food items and placing it within a parameter of an imaging system to be photographed from plural sides thereof.

Preferably, the first system comprises a track for moving the food items thereon to the food rack system, wherein the rack system comprises stacks of rotatable shelves, the rotatable shelves are ring-shaped and define a central hollow space, and at least one motor is provided for driving the rotatable shelves. The motor is located within said ring-shaped shelves. Rotation poles are located within the hollow space.

Preferably, the system includes a printing system for printing customer labels. Each food item has affixed thereto an identification tag that can be read to retrieve various parameters associated with specific food items, the tag information comprising one or more of a barcode, a visual code, an RF tag, and a Bluetooth chip.

Other features and advantages of the present invention will become apparent from the following description of the invention, which refers to the accompanying drawings.

### BRIEF DESCRIPTION OF THE DRAWINGS

FIG. 1 is an overall block diagram of the system's methodology that enables providing to the public online fresh food shopping portals.

FIG. 1a is an overall block diagram of the system's hardware subsystems that enable providing to the public an online fresh food shopping portal.

FIG. 2 is an illustration of a user information panel and accompanying video display for choosing individual fresh food items.

FIG. 3 illustrates, diagrammatically and perspectively, a shelf or rack system for the storing of food items thereon, together with the system for loading the shelves and for dispending food products from the storage shelves or racks.

FIG. 4 provides additional detail to the illustration of FIG. 3, in the form of conveying systems that deliver food products to the shelve or rack system, and also attends to the food item selections and delivery in accordance with the present invention.

FIG. 5 illustrates an embodiment that utilizes a rack system different than the one in FIG. 4.

FIG. 6 is a perspective diagram of a food rack system using individual robots, to store and retrieve each food item, in accordance with an embodiment of the invention.

FIG. 6a is a perspective of a container for a single food item that is used to ferry the food item to and from the storage system by the individual robots, in the FIG. 6 embodiment.

FIG. 6b is a perspective of the robot holding the food container of FIG. 6a.

FIG. 6c shows a single food rack used in the embodiment of FIG. 6.

FIG. 6d shows a floor plan for a multilevel rack system using the rack of FIG. 6c.

FIG. 6e depicts a robotic arm and a photography station to obtain multi-side images of each food item.

FIG. 6f is an exploded view of some of the components shown in FIG. 6e.

FIG. 6g is an exploded view of a component of the individual robot of the embodiment of FIG. 6.

FIG. 6h is a perspective of a turning device for turning individual food items, which is usable with the embodiment of FIG. 6.

FIG. 7 diametrically depicts a food testing station for obtaining parameters about the each food item, including images of the food item, its tactile characteristics and other freshness characteristics.

FIG. 8 is a flow chart of the overall process steps associated with the system of the invention.

FIG. 9 is a flow chart showing process steps involved in obtaining parametrical data relative to food products.

### DETAILED DESCRIPTION OF EMBODIMENTS OF THE INVENTION

In general, the main components of the system of the present invention comprise a shelve or rack system for food products; a robotic system for placing food items on the shelf system and withdrawing/placing food items into individual customer shopping boxes; a camera system for obtaining still and/or video footage of each individual food item for customer review; a conveyance system for delivering food products to the rack system and for withdrawing food items and packing the same in customer boxes and delivering such boxes to customers; and, finally, an overall system processor for controlling the physical hardware and for keeping track of customer orders, product inventories and the like, as well as various administrative system tasks.

From the algorithmic perspective, the system of the present invention uploads food items, scans the food items, processes, stores and transmits food item images to a display system for users, keeps track at all times of the locations of the food items, receives customer orders and processes such orders which involves placing a customer purchase box next to the shelving system, placing requested items in the box, creating customer information on the box or on the label affixed to the box, and conveying the box to the next location in the delivery system.

In the aforementioned overall algorithm, the uploading system comprises uploading food items from their original containers onto specific shelves using robotic equipment that is programmed to perform this action. In one embodiment, each item is placed on a motorized “plate” or shelve (a separate motor may be provided for each plate or shelf for food products or a single motor can drive multiple plates by providing rotational motion via a belt or gear or other transmission system).

In one embodiment, the scanning system is generally implemented by having food items scanned when they pass in front of a video camera and as the motorized plate rotates 360 degrees the camera images and/or creates a video or still photos of each foot item. All images or the video are stored in a database with a number that represents the exact location of the item and all this information is transmitted to a database for the purpose of complete tracking of all food items at all times. The machine needs to keep track all the time which location number is now in front of the camera. There are multiple manners as to how to perform this action as more fully described below. One way is to use a stepper motor to rotate the shelves, so that the controller or overall processor knows the angular positional orientation of each plate. Another way is to use an encoder or optical encoder or a tactile sensor or a barcode or a Bluetooth system, etc.

Finally, all the processing is accomplished via the conveyance system using a robotic system and a box or another form of packaging for fulfilling a customer order and loading all this information for downstream processes that result in the fresh food items rapid shipment (within an hour or so) to the customers.

The various Figures of the present specification are intended to depict various components and sub-components of the present invention, in several versions thereof. Indeed, the preferred embodiments are described below, in the chronological order of the figures.

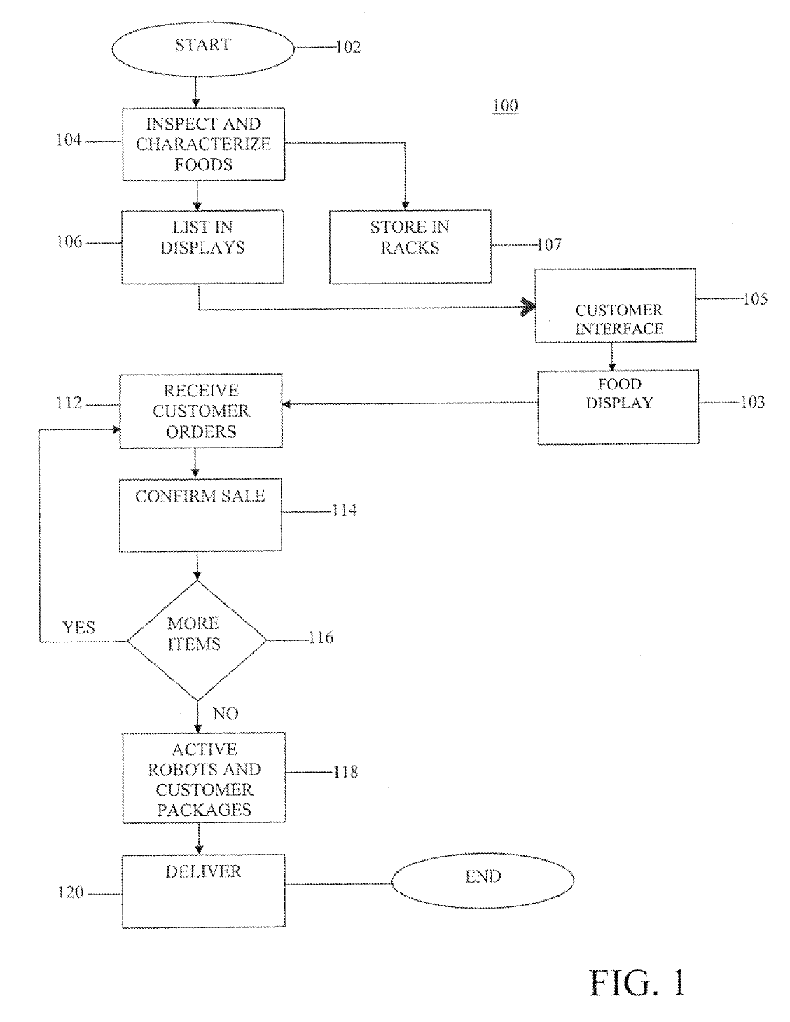

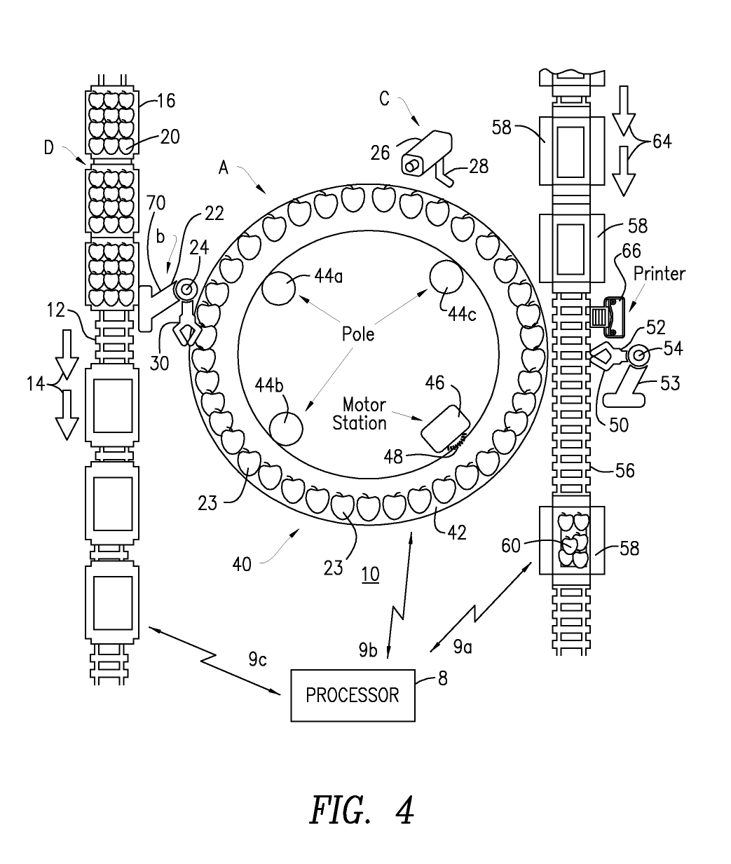

Referring to the Figures, the block diagram of FIG. 1 illustrates, in accordance with one embodiment, the overall algorithmic process including the start module 102 in which the program is initialized and updated, in the usual manner as would be readily understood by one of ordinary skill in the art in the programming of a control system that involves vendors and purchasers. At module 104, the processor receives incoming, just delivered food items and categorizes each item by assigning a unique code thereto. At module 106, the overall processor (processor 8 in FIG. 4 which communicates generally with the hardware either by hardwire connections or wirelessly as indicated by reference numerals 9a, 9b and 9c) causes the system to inspect each food item, characterize its parameters such as by images (still and/or video), weight, volume, freshness, smell, softness, prices, date/time of inspection, etc., and stores this information at appropriate locations which are indexed relative to each food item.

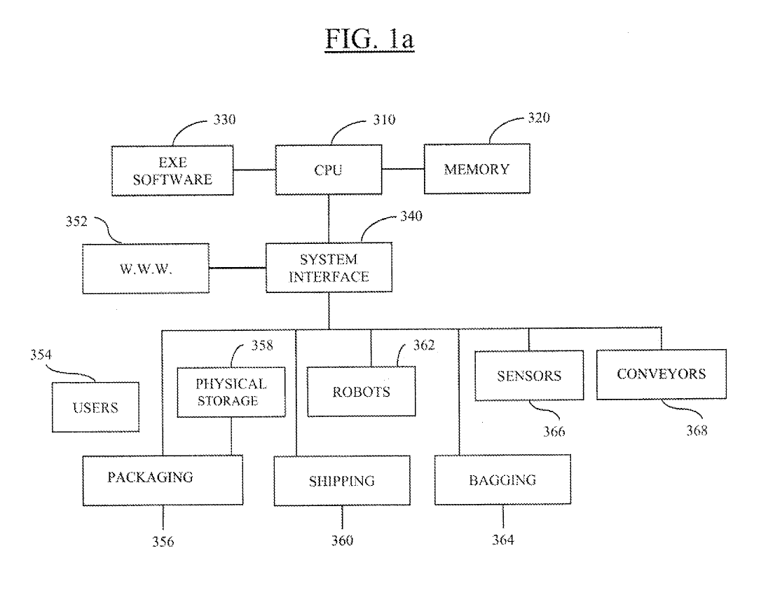

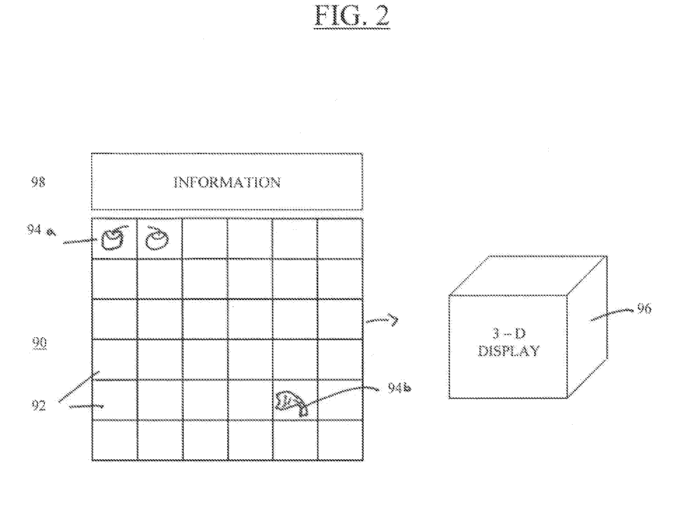

Once the food item characteristics have been determined, the process continues at module 107, at which the processor 8 directs robotic arms to lift and place or otherwise convey each food item to a specific location on the shelving or racking system of the present invention and subsequently, in software module 108, the item image is stored to be eventually displayed in a display system that is more specifically described below, relative to FIG. 2.

At this point, customers who have viewed the display system and know precisely which food product categories, and more specifically food items, they wish to buy may proceed to software module 110, which inputs the specific customer orders for the system to begin the retrieval of the specific food items and the placement thereof in the customer's shipping box.

The overall system provides a module 105 that allows customers to initially search the overall system to identify in the system the specific items the customer is interested to inspect, their price ranges, the quantities available and other characteristics, which causes the software to then display at module 103 a display of food items that the customer expressed a desire to see and select food items from. In conjunction therewith, one would appreciate that in a highly developed vending system there might be hundreds of different products to select from, at various price ranges and many other criteria that would make it impractical to have a display system that has all of the food items shown on a single screen or even a series of screens. In other words, the software narrows the display of food items based on filtering information inputted by the purchaser.

Referring now to software module 112, at each state the customer can view a personal shopping basket that shows the food items in the basket and the customer may confirm the item choices in this basket as indicated in module 114. Continuing to decisional box 116, the software queries whether additional items are desired to be selected. If the response is yes, then the program returns to module 110 to receive additional instructions or selection choices from the customer. However, if the response is “no,” indicating that the customer shopping session has been completed, the program proceeds to module 118 at which the overall processor 8 directs the system to activate the robotic conveyors of the system to then either complete the process by causing the conveyance system to ship the box further downstream in the order processing, to the shipping/loading docks of the vendor.

In another embodiment, the packing of a customer food box does not commence until all the items have been selected. Only after the customer provides a final approval, are items uploaded from the rack system, into the individual box of each purchaser.

Various overall system options are available. In accordance with one alternative, each item that has been selected by a customer will only be held in a “selected” state for a predetermined time, for example 10 or 15 minutes, during which it is not available to any other customer to be placed in their shopping basket. However once that time period has elapsed, the food item is released back to the general availability database.

In accordance with an embodiment of the invention, the system of the invention provides a gripping mechanism with a tactile sensor to measure the “softness” of the food. This can be done by applying a squeezing force and sensing the food items' “resistance” to deformation, as a “softness” parameter. Another parameter may be provided in the form of a smell signature, for example by using a spectrometer or the like to take a “whiff” of each food item and record either a graphical or other visual indication where customers may come to learn to associate with preferences. Odor assessment is usually performed by human sensory analysis. But in the mechanized system of the invention it is performed chemosensors or by gas chromatography. The latter technique gives information about volatile organic compounds but the correlation between analytical results and actual odor perception is not direct due to potential interactions between several odorous components. The invention uses an output of these devices and displays it graphically or in any visual manner. Thereby, customers who has purchased in the past say tomatoes, and been provided with a visual signature of its odor/smell, will be able to look for that same or similar visual smell/odor graph on their next purchase of the same food items.

The concept is to mimic human olfaction, whereby the system functions as a non-separative mechanism: i.e. one in which an overall odor/flavor is perceived as providing a global fingerprint. Essentially the instrument consists of a sensor array and pattern recognition modules, that generate unique signal patterns to characterize specific odors/flavors. The prior art is knowledgeable on this subject as can be appreciated from reviewing: https://en.wikipedia.org/wiki/Electronic_nose.

One of skill in the art would also appreciate that the manner of handling food items can be either by using a robotic system with forceps arms or a ladle system that scoops the item or a vacuum system that holds each item by suction and that there are myriad other ways that would be apparent to one of ordinary skill in the art. Regardless, once the purchase session has ended, the package being sent downstream for being delivered is handled/processed at module 120.

Referring now to FIG. 2, in general, a customer would see a grid-like display with a single unique item in each grid location. For example, in this figure it might be an apple where one is actually seeing the hue or the other aspects of the apple, when placing the mouse curser over the desired item. Also, the item may be rotatably displayed in live fashion as a 3-D display 96, to the side of the display, in a manner familiar in the art. Optionally, the display 90 might depict apples 94a in one row and meat steaks 94b in another row of the display, and so on.

Thus, the overall display 90 has individual grid boxes 92 each depicting one food item and an information box 98 is provided where general information pertaining to each item such as its brand name, price per ounce, weight, size, softness and/or smell parameters are listed, in correspondence to the item that the customer points to by means of a cursor or by placing a finger on a touch screen, etc.

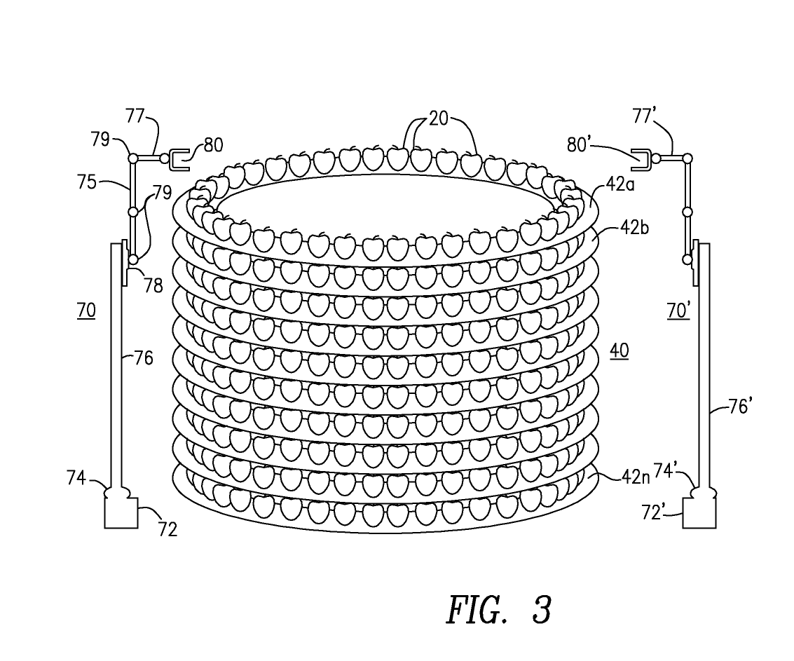

Referring to FIG. 3, the shelving or rack system 40 may be implemented in the form of individual plates 42a, 42b . . . 42n, where on each shelf the food items 20 are organized in a manner that makes it easy for the robotic system 70 to deliver the food items and place them on the racks and/or for the robotic system 70′ to withdraw individual items and pack them into customer boxes' shopping boxes.

More specifically, the incoming food robotic system 70 may comprise a base 72, a swivel joint 74, a tall upright arm 76, and a robotic grasping arm consisting of a base 78 that can either be rotatable or even travel in a track up and down the upright arm 76 and various control arms 73, 75, 77 joined by a swivels 79, ultimately controlling a forceps 80 that can move in 3-D to reach any food item on the rack system 42, to place or put item thereon.

Although not shown in this figure, the entire rack system can be rotated in unison so that the robotic system 70, 70′ would always be at one angular position relative to the rack system.

Juxtaposed to the food item delivery robotic system, the withdrawing or fulfillment robotic system 70′ similarly comprises a base 72′, a swivel on 74′, a main upright shaft 76′ together with various manipulating arms 77′ and the forceps 80′, that operate as described above.

Although in FIG. 3 only two robotic arms systems are illustrated, in fact, the system can be constructed in a size and dimension large enough that multiple incoming food handling robotic systems 70 are provided over a stretch of 180 degrees of the system, and multiple robotic systems are provided in an outgoing robotic system 70′, extending over 180 degrees. Alternatively, the robotic arms may be interspersed with one another, to increase tremendously the overall throughput of the system in a manner that should be instantly apparent for one of ordinary skill in the art.

Referring to FIG. 4, in the top view therein, the rack system 42 shows the food item 20 being received in individual traveling cartons 16 moving on tracks 12, so that the adjacent robotic system 70 with components 22, 24 and 30 enable unpacking the box 16, place the items on the racking or shelf system 42, with empty boxes being shipped away to be refilled and returned.

While the racks 42 are being rotated under control of the processor 8, using the motor 46 with its gearing system 48 and support shafts 44a, 44b and 44c, the camera or scanner 26 which is supported preferably on a swivel handle 28 can photograph each food item from various angles or even from above, depending on the camera movements, to store that information for purposes of customers reviewing the same. Thus, the overall rack system 40 with said individual location cells 42 could be loaded with various food items and these food item images and other characteristics are stored in the aforementioned customer display database that appear on their individual workstations or computers when they access the vendor's system for purchasing items.

To fulfill the orders, the outgoing conveyor system comprises a track 56 on which empty customer boxes 58 travel in the direction of arrow 64 whereby when they appear adjacent to the robotic arm system 52 comprising the components 53, 54 and forceps 50, individual items are grasped and placed into the boxes, for example the food item 60. The printer 66 prints up various labels that go on the box, and then these full boxes travel downstream to ultimately be shipped to the customer.

The stacking of food items as described by reference to FIGS. 3 and 4, represents one preferred embodiment of the invention. But it should be rather apparent to people of ordinary skill in the mechanical arts that many stacking and racking systems as well as conveying systems can be used for the purposes of the invention. Thus, for example, and with reference to FIG. 5, one can appreciate that the racking system can comprise an immovable system with individual boxes 152a, 152b . . . 152n, with bins for food items stored in boxes. These boxes are vertically separated by a distance that allows food to be inserted/loaded via their permanently open tops. When an order for a particular item has arrived, the bottom door for example 153 of a particular location opens and the food item drops onto a slightly inclined and very smooth shelf, which is shaken whereby the food travels to the edge of the inclined shelf 154a and from there picked up by the robotic system shown in FIG. 4, to be delivered and placed in a customer's purchasing box.

Rather than restocking these boxes 152a . . . 152n individually, a moveable box 156 can travel in between the boxes as well as up and down and at once fill an entire box with food items. Thus, for example if the box 152 has only two food items left, it is possible for the system to drop those two items, retrieve them and return them to the incoming rack and then fill the entire box 152 at once with food items, to realize a more efficient food item handling system.

In general, more detail-oriented, useful information is included in the actual physical implementation of the invention. For example, each food item may contain a label that provides a unique code whereby when a food item is picked up, reading the label providing more information about it. Alternatively, the food item can be recognized by having a barcode or near-frequency tag (NFC) attached thereto, with the near-frequency tag being removed before the food item is placed in a customer basket. Additional information might be provided to customers as to how long a particular food item has sat in the rack system, whereby some customer might not want to purchase a tomato that has been sitting on the shelf for 3 days. Other information about the processing of fresh food items is described in U.S. Pat. No. 6,539,781 and the contents of said patent are incorporated by reference herein.

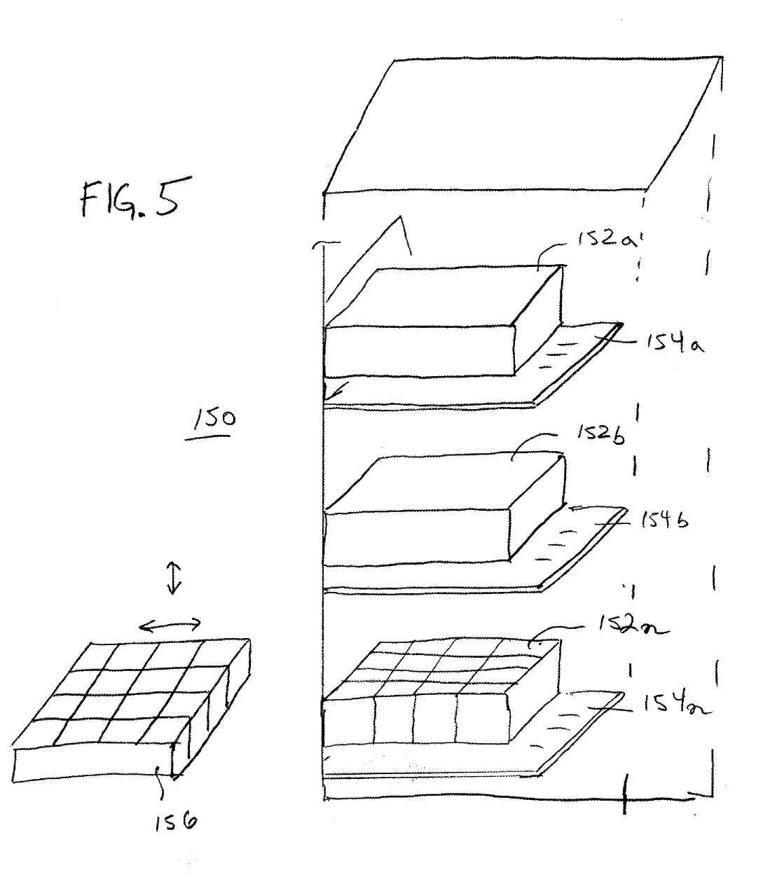

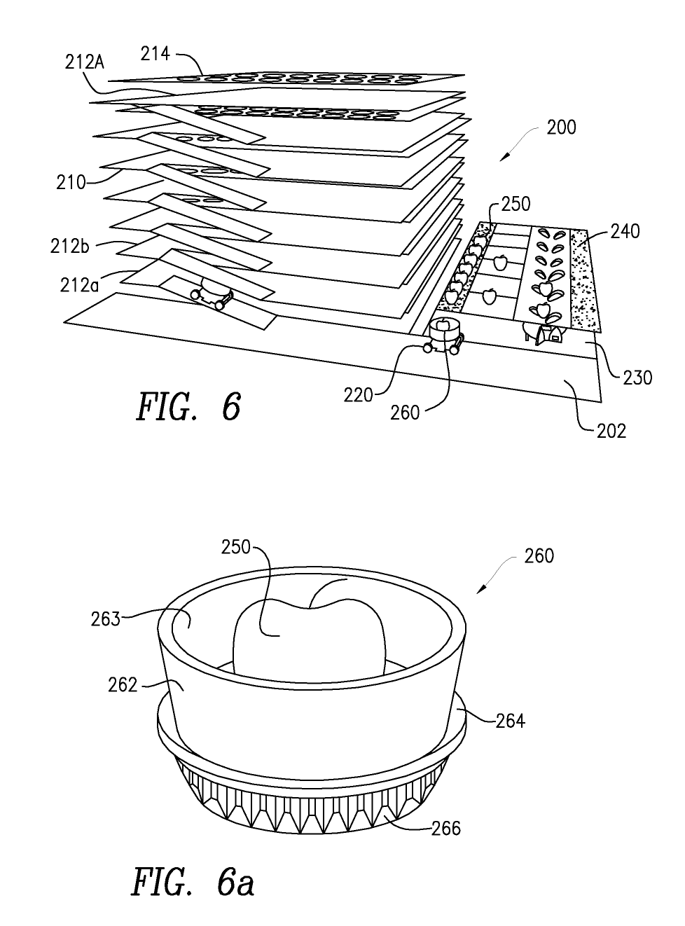

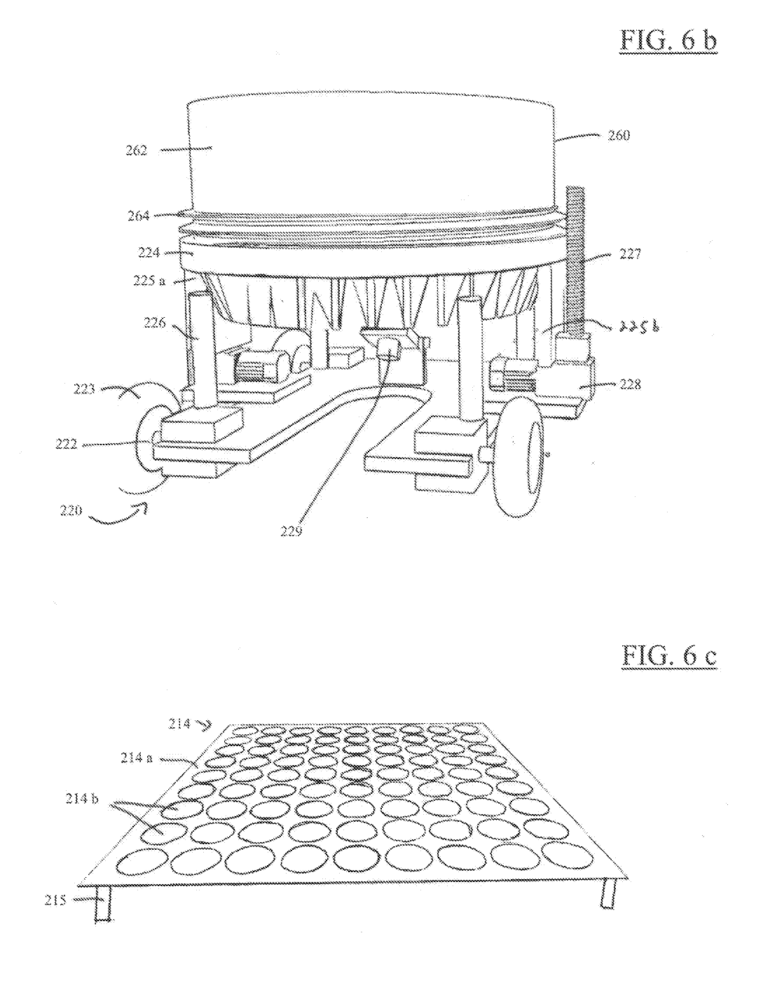

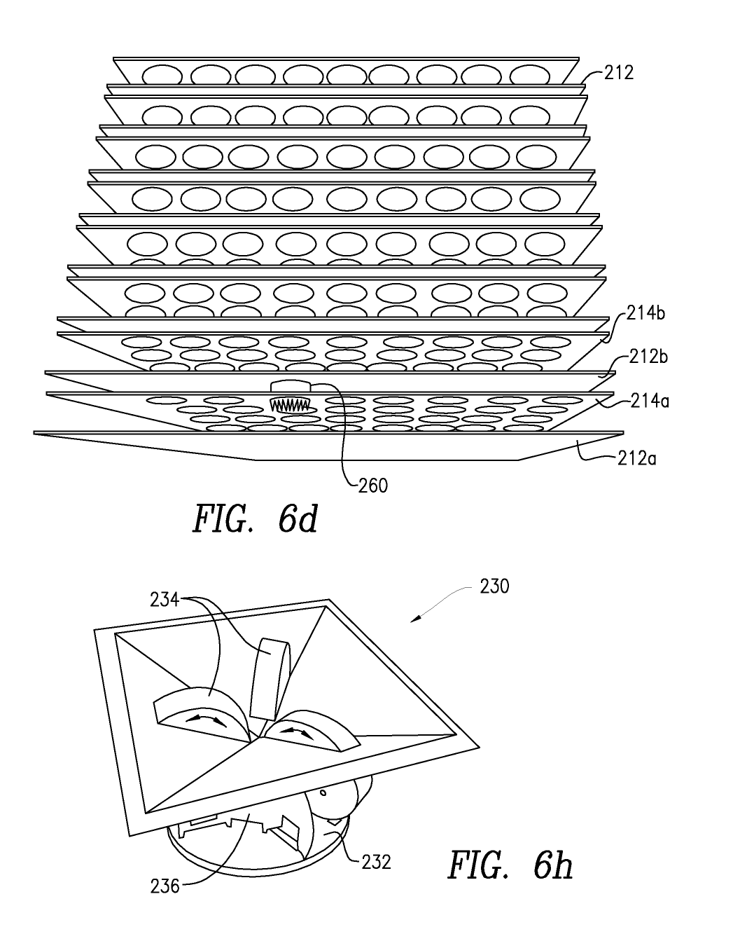

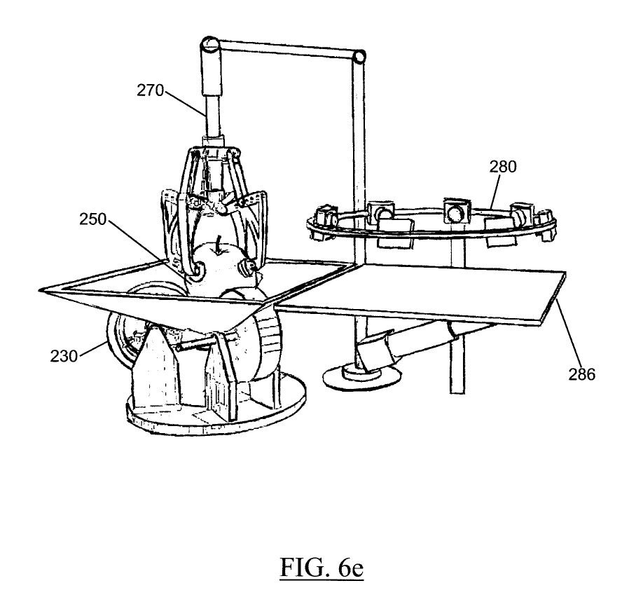

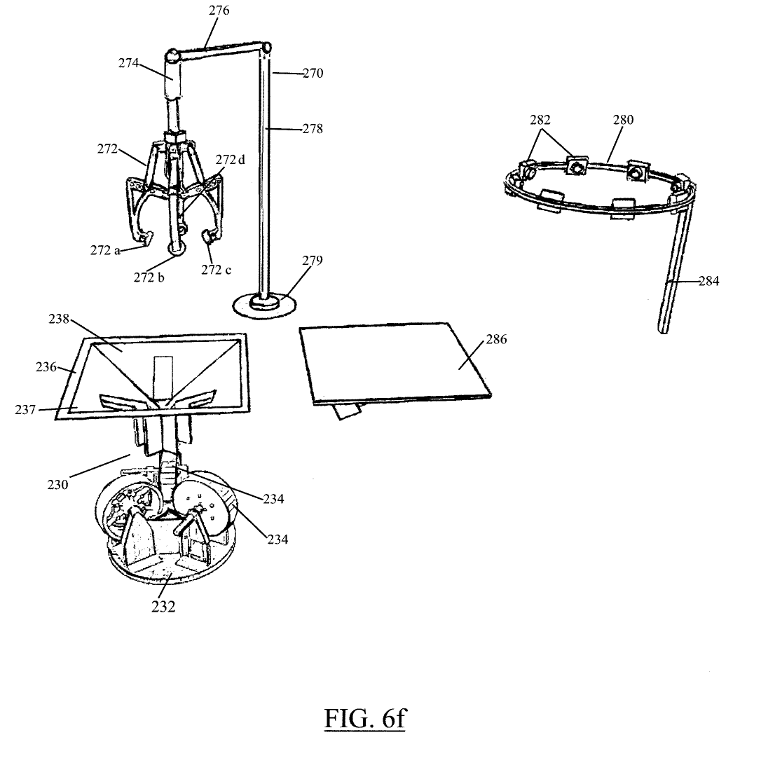

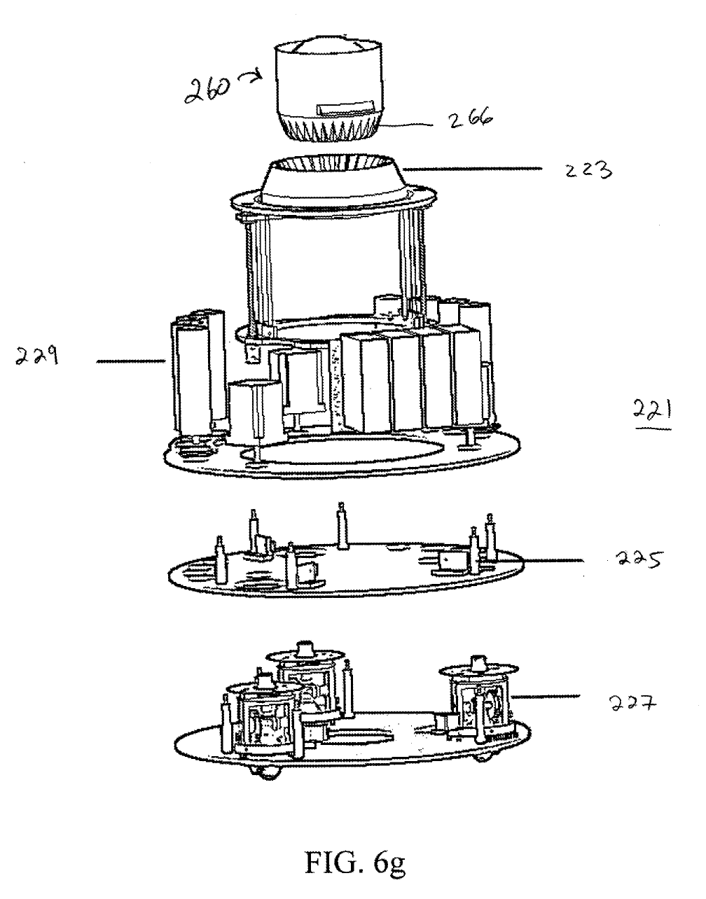

Turning to FIG. 6 and its accompanying FIGS. 6a-6h, in accordance with a particularly preferred embodiment of the invention, a system is described which processes every piece of produce individually, and tests it for ripeness and other parameters and also images each item and thereafter sequences the uploading of each item and its parameter data into a database, made available at a customer website portal.

In general, the below-described system is intended to be modular, which enables the system to be scaled in size to handle different size businesses, having different budgets. Thus, the overall food item testing, storing and user selection system 200 comprises a food item rack system 210 which is multileveled and includes a rampway 204 at each “floor” of the rack system, on which individual robots 220 can travel to reach each floor level to carry, store and selectively retrieve food items 250, from and to a staging area 240. The system 200 also has individual food item rotators 230 which enable each food item to be rotated from side to side and forward and backward to be allowed to be imaged from all sides. The robots 220 are designed to carry individual food item containers 260 (FIG. 6a, FIG. 6b) and to store each food item in its respective container 260 on individual racks 214 (FIG. 6c), where each of the containers 260 is supported by one of the container circular slots 214b which are defined in the body 214a of the shelf 214.

With reference to FIG. 6d, each of the individual racks 214a, 214b . . . 214n is supported above a floor, 212a, 212b, etc., with enough space for the robots with the container located on them to travel on the floor and to raise the container and tread it into the opening in the rack 214 above it, as illustrated in FIG. 6d. In this manner, each robot 220 picks up a container 260 with a food item 250 therein, travels over the main storage path 202 and then unto the first ramp 204, then over the first floor 212a to a designated position on that floor underneath the shelf 212a, as has been directed by a central computer, e.g., the CPU 310 (FIG. 1a). At the precise location, the robot 220 raises the container 260 and turns it one or two turns to be threaded into the openings 214b. Naturally, the robots proceed to any of the floors as directed. Preferably, a corresponding set of ramps (not shown) are located on the opposite side in FIG. 6 to allow robots to enter the storage structure 200 through one entranceway and to return via another path, whereby robots can follow one another closely without colliding or interfere with each other.

Referring again to FIG. 6a, the typical container 260 has a container body 262 with a rim 264 and a serrated bottom 266, which enables its rotation. The interior 263 is designed to hold a food item 250. As shown in FIG. 6b, the robot 220 has a chassis 222 with four wheels 223, a circular rim 424 supported on opposite sides by beams 225a, 225b that rest on motors 228, which turn the screw 27, causing the beams (225a, 225b) to rise and thereby lift the rim 224 higher to change the elevational position of the container 260, so it could reach the rack 214 above it. The camera 229 allows the robot 220 to navigate the robot along pathmarks or embedded coils (not shown) in the floors to reach any given destination which is provided by the computer, over a grid pattern that divides the storage space into a grid pattern. Thereby, the robot 220 can travel (as instructed by a computer) to specific floors of the storage rack 210, and to specific positions underneath a specific location 214b in the floor 214a of 214a. The beams 215 under each rack 214 are of a height that enables the robot 220 with its container and food item, to fit and travel on each floor without obstruction.

FIG. 6 illustrates the staging area having the rotators 230. An individual rotator as shown in FIG. 6h comprises rotatable wheels 234 located over a base 232 and rotated selectively by a driving mechanism 236. Thereby when a food item, for example an apple, is placed on the rotator 230, it can be rotated from right to left, forward to backward and thereby be imaged from different directions, thereby enabling the storage of photos of the precise appearance including color, surface texture, providing high resolution images for customer review.

FIG. 6f shows several separate components, which are shown and integrated with one another in FIG. 6e. These components include the aforementioned rotator 230 which has an upper container structure 236 with downwardly sloping surfaces 237 which define an interior 238. The rotatable wheels 34 fit through the openings in the holder and are supported in a base 232. They are rotated to be able to turn a food item, for example an apple, from side to side as previously described.

The gripper 270 has a gripper section 272 with several, preferably at least four arms 272a, 272b, 272c and 272d, which is supported by a telescoping arm 274 which is spaced by a lateral member 276 from an upright 278, that is anchored by a base plate 279 to a floor. The gripper 270 can be placed over the plate 236 to pickup a food item, place it on a base 286 and in that position the imager 280, which has surrounding cameras 282 and which is supported on a post 284, can photograph the food item from all directions, all as can be appreciated from FIG. 6e.

FIG. 6g provides further detail about a robot 221 which is shown in an exploded view in this figure. The robot 221 has a container holder 223 with an inner serrated surface that matches the serrations 266 on the container 260. Thereby, when the robot 221 turns on its axis, it is able to turn the container 260 clockwise and counterclockwise, to obtain the functions previously described. The robot 221 comprises several motors 227 that can turn its wheels in all directions to either move forward, backward, sideways and/or rotate. The solenoids 225 assist in raising the container holder 223 and works in this connection with the supports 229.

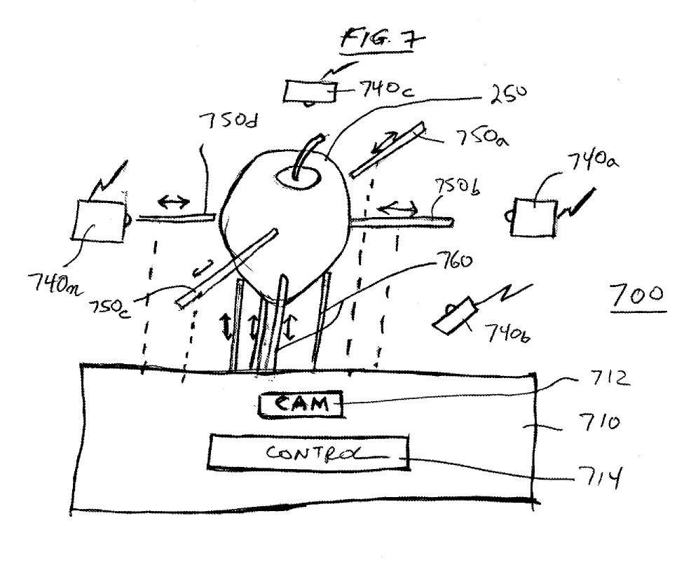

Referring to FIG. 7, the objectives of the invention to provide a “softness” parameter for the food items, can be realized with the system 700 that has a base 710 with various electronics including a camera controller 712 and an overall controller 714, which operate various holding fingers 760 that can raise the load 250, i.e. the food item, to be firmly held without moving, while tactile fingers 750a, 750b, 750c and 750d are able to be moved toward the food item 250 to obtain the softness parameter. Typically, the system knows what type of an item is held by the fingers 760 and is able to move the tactile fingers 750a toward the food item until they touch it. Thereafter, a given force is applied and the amount of movement is sensed, to obtain a measurement that is indicative of the softness of the food item.

The camera controller 712 controls several cameras, 740a, 740b, 740c . . . 740n that can be used for obtaining the various images of the food items as previously described.

The computerized overall control system 300 includes the needed hardware and software components, for example, as depicted in FIG. 1a. This computer/software system 300 includes a central CPU 300 that carry as various instructions that executes various instructions and handles data that can be stored in the general memory and process based on executable program files, i.e., executable software 330.

The CPU 310 interfaces with the overall system's hardware and software components through a system interface 340 which allows the overall system 300 to direct the functioning, and to send and receive information through such components as a worldwide web (WWW) 352 interface through which various users 354 can be reached and communicated with. The system interface 340 is also in communication with the various system robots 362 its on parameter sensors 366, the conveyors 368, the physical storage 358 as well as the packaging subsystems 356, the shipping system 360 and/or the bagging system 364.

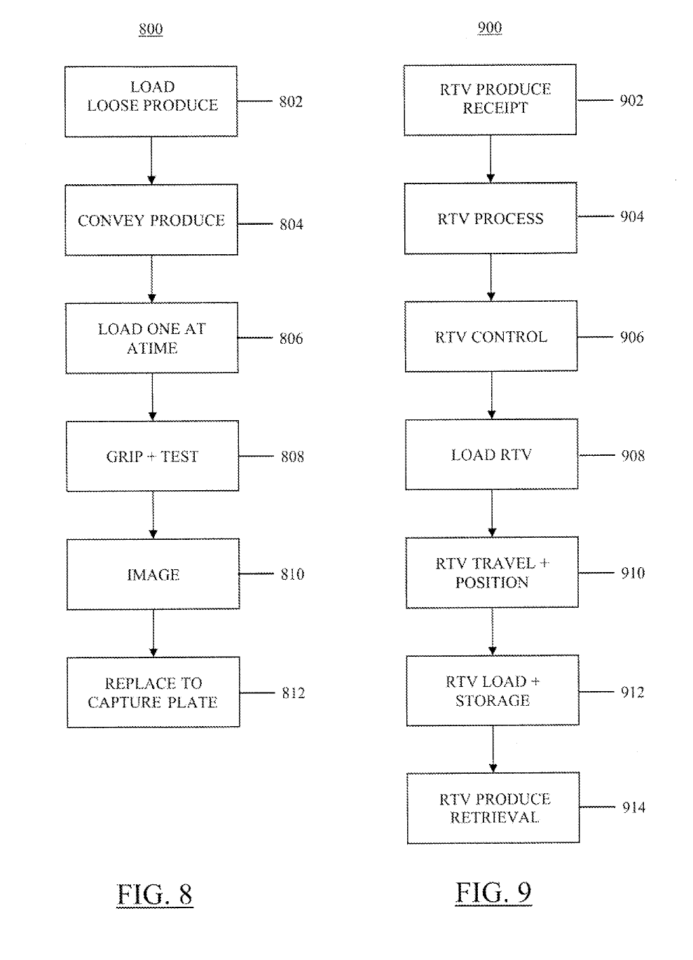

With reference to the overall system flowchart of FIG. 8, the overall system process 800 comprises a load loose produce module 802. In this module, humans place a bunch of loose produce onto the initial loading station. The loading station is then tilted towards a roller conveyor that slowly starts releasing five to ten fruits at a time onto the roller conveyor. In the convey produce process 804, the roller conveyor continues to transport the produce onto a singling conveyor, that runs at a higher speed to ensure that the fruits are spaced from one another. In this stage, the fruit continues to proceed to following conveyors that maintain the fruit separated from each other.

In the load one-at-a time process 806, one single fruit lands at a time onto a positioning plate or container, which positioning plate positions the fruit in an upright position to prepare for imaging. In the next grip & test process 808, a robotic arm places its grips over the fruit and pressure tests it for testing its ripeness grade.

In the next imaging process 810, the robotic arm lifts the fruit and places it on an imaging plate and imaging cameras capture at once several images, for example, eight images, and sends those images to a main server for processing and creating a 360° view of the fruit.

In the final overall process, the release to capture plate process 812 releases the fruit from the imaging capture plate via automatic tilting of the plate, which releases the fruit onto a conveyor which then queues the fruits picked up by the robotic vehicle to be transported to its storage cell.

In one embodiment, the invention utilizes an RTV transport vehicle and the overall function of this vehicle is explicated below with reference to FIG. 9 that shows its overall system process 900. The RTV system process 900 commences at the RTV produce receipt process 902. At this process, the RTV transport vehicle receives the fruit and travels to the storage cell's area to place the fruit in an available cell. The RTV is also responsible to pick up the fruit when the fruit has been purchased for delivery and delivers it to an order fulfillment area.

In the next RTV process 904, the commands to the RTV are processed. Subsequently at the RTV control process 906, the RTV receives commands from the central service to pick up a given fruit from the post-imaging conveyor for storage. The process then proceeds to the load RTV process 908, at which the RTV receives at a plate located on the top thereof from the loading conveyor.

The RTV process then proceeds to the RTV travel and position process 910, at which, the RTV travels to a given storage cell, using various position technology to guide its way to the storage cells and then positions itself directly under a particular food item. The process then proceeds to the RTV load and storage process 912, at which the plate of the RTV is raised to its highest position to reach the storage cell and the plate on which the fruit is placed is rotated into a storage cell by twisting the entire RTV by 90° which results in the plate being locked into the produce cell.

In another RTV process, namely in the RTV produce retrieval process 914, the RTV responds to a command from the central computer to transport a fruit to a fulfillment area. The RTV then travels to the storage cell, raises its receiving plate to touch the plate or the container in which the fruit is located, and the RTV then counter rotates by 90° to twist and release the fruit and to transport it to the fulfillment area.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. An online vending system for vending physically unique food items (FUFI), the vending system comprising:
a food rack system configured to hold a changing inventory of the physically unique food items, said food rack system being configured to store therein a plurality of said food items in a configuration that allows on-going replenishment of said food items;
a first system for conveying the food items to and from the rack system;
a second system for retrieving and shipping customer-selected ones of said food items for being shipped to customers;
a processor for controlling an overall operation sequence of said online-vending system, said processor being configured to provide to remote customer electronic devices at least one customer display of said food items, each food item being displayed individually and having parameter data pertaining specifically to said each food item displayed relative to said each item, in a manner that enables the customers to select desire ones of said food items shown on said customer display and based on said parameter data;
a processing facility that enables customers to communicate, via a user interface, to the processor to select specific categories of the food items to be viewed on a screen of the customer's electronic device and to thereafter select specific food items based on said parameter data provided on the screen relative to each food item, said parameter data visually or alphanumerically identifying one or more of a visual appearance, color, size, price/weight and/or smell/flavor data for each of said food items; and
a robotic system for receiving various food items selected by a customer and for controlling the packaging and the shipping thereof via said second system to the customer.

2. The system of claim 1, wherein the first system comprises individual containers for carrying therein each of the physically unique food items and a plurality of individual robots each configured to hold one of the containers and to convey each container to and from the rack system based on receiving specific instructions from the processor.

3. The system of claim 2, wherein the rack system comprises a plurality of vertically stacked platforms, each platform having a floor space for the individual robots to travel thereon and including a stacking ceiling into which the containers carried by the individual robots can be inserted by being raised and attached to said ceiling, and said individual robots being able to travel on ramps to reach any of said platforms.

4. The system of claim 3, including a tool for holding a food item and being able to rotate and change the orientation of the food item and including an imaging system for photographing the food item with said rotating device.

5. The system of claim 1, including a gripping mechanism and an imaging method mechanism and the gripping mechanism being configured to raise individual food items and placing it within a parameter of an imaging system to be photographed from plural sides thereof.

6. The system of claim 1, wherein the first system comprises a track for moving the food items thereon to the food rack system.

7. The food vending system of claim 1, wherein the rack system comprises stacks of rotatable shelves.

8. The food vending system of claim 7, wherein the rotatable shelves are ring-shaped and define a central hollow space.

9. The food vending system of claim 8, including at least one motor for driving the rotatable shelves.

10. The food vending system of claim 9, wherein said at least one motor is located within said ring-shaped shelves.

11. The food vending system of claim 10, further including rotation poles within said hollow space.

12. The food vending system of claim 1, for including a first robotic system for moving food items from said first conveyer into said rack system.

13. The food vending system of claim 12, further comprising a second robotic system for retrieving selected food items from said rack system and moving said food items to said second conveyer system.

14. The food vending system of claim 1, further including a printing system for printing customer labels.

15. The food vending system of claim 1, further comprising a camera system for photographing said food items and for providing photographs available on said customer displays.

16. The food vending system of claim 15, wherein said camera operating system is effective for providing video footage of said food items from different angles thereof.

17. The food vending system of claim 1, wherein each item has affixed thereto an identification tag that can be read to retrieve various parameters associated with specific food items.

18. The food vending system of claim 17, wherein the tag information comprises one or more of a barcode, a visual code, an RF tag, and a Bluetooth chip.
